(19) United States
(12) Patent Application Publication
(10) Pub. No.: US 2024/0100127 A1
(43) Pub. Date: Mar. 28, 2024

(54) TIRZEPATIDE THERAPEUTIC METHODS

(71) Applicant: Eli Lilly and Company, Indianapolis, IN (US)

(72) Inventors: Mathijs Christiaan Michael BUNCK, Indianapolis, IN (US); Laura FERNANDEZ LANDO, Carmel, IN (US); Jeffrey Scott RIESMEYER, Fishers, IN (US); Angel RODRIGUEZ BERNARDINO, Madrid (ES)

(21) Appl. No.: 18/546,477

(22) PCT Filed: Feb. 2, 2022
(86) PCT No.: PCT/US2022/014957

§ 371 Date: Aug. 15, 2023

Related U.S. Application Data
(60) Provisional application No. 63/150,187, filed on Feb. 17, 2021.

(51) Int. Cl. A61K 38/26; A61P 3/10

(52) U.S. Cl. CPC A61K 38/26; A61P 3/10

(57) ABSTRACT

An embodiment of the invention relates to method for increasing HDL-C in a patient in need thereof. An embodiment of the invention relates to a method for decreasing high blood pressure. An embodiment relates to a treatment method for a patient with refractory type 2 diabetes to provide normal HbA1C glycemia.

The present invention relates to the field of medicine. Provided are treatment methods for refractory type 2 diabetes (T2D) in patients failing to reach recommended glucose targets with metformin and SGLT-2 treatment. Provided are methods relating to increasing HDL-C levels in a patient in need thereof. Provided are methods for lowering blood pressure in a patient in need thereof.

### DESCRIPTION OF THE FIGURES

FIG. 1 graphically illustrates HDL-C increase using an efficacy estimand observed at 52 weeks in a clinical trial conducted substantially as described by Example 1.

FIG. 2 graphically illustrates HDL-C increase using an efficacy estimand observed at 40 weeks in a clinical trial conducted substantially as described by Example 2.

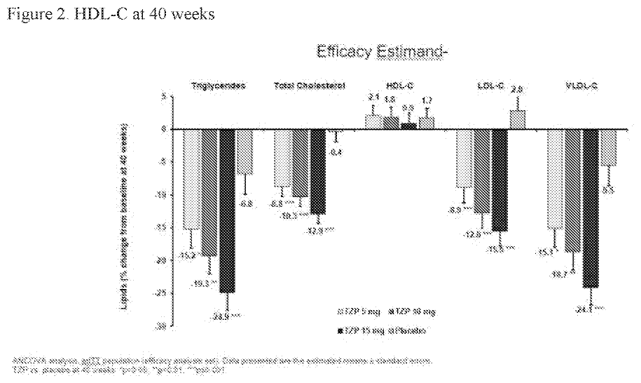

FIG. 3. graphically illustrates reduction in systolic blood pressure in a clinical trial substantially as described by Example 1.

FIG. 4. graphically illustrates reduction in diastolic blood pressure in a clinical trial substantially as described by Example 1.

Glycated hemoglobin (HbA_{1c}) is considered a key marker of glycemic control in diabetology. The American Diabetes Association (ADA) guidelines indicate that a patient with HbA_{1c} less than or equal to 5.7% is considered normal glycemia. Patient treatment goals for HbA_{1c} may vary by patient; however, persistently poorly controlled HbA_{1c} contributes disproportionately to the development of complications associated with diabetes. Patients with type 2 diabetes often fail to achieve normal glycemia despite treatment using the ADA treatment paradigm. Despite not within normal range, the ADA guidelines suggest a reasonable HbA_{1c} treatment goal of less than or equal to 7% following current treatment options of diet, exercise, metformin, oral diabetes treatments, followed by basal insulin. However, many patients fail to reach their HbA_{1c} goals despite clinical treatment and are considered to have refractory type 2 diabetes.

Refractory type 2 diabetes is generally explained by an insulin secretory defect, or beta cell damage, becoming more and more profound with time, against a background of (relatively stable) insulin resistance. Patients living with type 2 diabetes for at least 8 years are more likely to suffer from refractory type 2 diabetes. Therefore, there is a desire for a treatment method providing normal, or near normal glycemia, in a patient with refractory type 2 diabetes. There is a desire for a treatment method providing normal, or near normal glycemia, in a patient with refractory type 2 diabetes, wherein the patient has been treated for type 2 diabetes at least 8 years.

Nearly half of American adults have high blood pressure. High blood pressure is a risk factor for stroke, coronary heart disease (CHD), and other significant health threats. Many patients with type 2 diabetes and obesity experience high blood pressure; however, approved treatments for diabetes typically have little or no effect on controlling high blood pressure. There is a desire for treatment options to manage high blood pressure. There is a desire for a method to treat high blood pressure in a patients with diabetes.

Low serum levels of high-density lipoprotein cholesterol (HDL-C) is another known risk factor for coronary heart disease (CHD). Patients with type 2 diabetes often experience low serum levels of HDL-C; however, approved diabetes treatments generally fail to raise HDL-C. Longitudinal population studies have confirmed that HDL-C is inversely and independently associated with the risk of developing CHD. There is a desire for a drug therapy to increase HDL-C levels. There is a desire for a treatment method to raise HDL-C in a patient with type 2 diabetes.

The present invention provides methods for treating, preventing, or delaying high blood pressure, comprising administering tirzepatide, or a pharmaceutically acceptable salt thereof. The present invention further provides methods for treating, preventing, or delaying high blood pressure in a patient diagnosed with type 2 diabetes, comprising administering tirzepatide, or a pharmaceutically acceptable salt thereof.

The present invention provides methods for treating, preventing, or delaying low HDL-C, comprising administering tirzepatide, or a pharmaceutically acceptable salt thereof. The preset invention further provides methods for treating, preventing, or delaying HDL-C in a patient diagnosed with type 2 diabetes, comprising administering tirzepatide, or a pharmaceutically acceptable salt thereof.

The present invention provides methods for treating, preventing, or delaying refractory type 2 diabetes in a patient having type 2 diabetes for at least 8 years, comprising administering tirzepatide, or a pharmaceutically acceptable salt thereof.

The present invention provides methods for treating refractory type 2 diabetes in a patient having type 2 diabetes for at least 8 years, comprising administering tirzepatide, or a pharmaceutically acceptable salt thereof.

In an embodiment the patient in need of treatment for refractory type 2 diabetes has an HbA_{1c} of greater than 10%.

In an embodiment the patient in need of treatment for refractory type 2 diabetes has an HbA_{1c} of greater than 11%.

In an embodiment the patient in need of treatment for refractory type 2 diabetes has an HbA_{1c} treatment goal of 5.7%. In an embodiment the patient in need of treatment for refractory type 2 diabetes has an HbA_{1c} treatment goal of less than or equal to 5.7%.

In an embodiment the patient in need of treatment for refractory type 2 diabetes has an HbA_{1c} treatment goal of 6%.

In an embodiment the patient in need of treatment for refractory type 2 diabetes has an HbA_{1c} treatment goal of 7%.

The present invention provides methods for treating refractory type 2 diabetes in a patient who is non-responsive to metformin, SGLT-2 or metformin plus an SGLT-2 inhibitor, comprising administering tirzepatide, or a pharmaceutically acceptable salt thereof.

Accordingly, an embodiment provides a method for treating, preventing, or delaying high blood pressure in a patient in need thereof, comprising administering an effective amount of tirzepatide or a pharmaceutically acceptable salt thereof, to the patient once weekly. A further embodiment, provides a method for treating high blood pressure in a patient diagnosed with type 2 diabetes, comprising administering an effective amount of tirzepatide or a pharmaceutically acceptable salt thereof, to the patient once weekly.

In another aspect, the present invention provides a method of preventing or delaying high blood pressure in a patient, comprising administering tirzepatide in a therapeutically effective amount to the patient once weekly.

In another aspect, the present invention provides a method of preventing or delaying high blood pressure in a patient diagnosed with type 2 diabetes, comprising administering tirzepatide in a therapeutically effective amount to the patient once weekly

In another aspect, the present invention provides use of tirzepatide for the preparation of a medicament for treating, preventing or delaying development of high blood pressure in a patient, wherein the medicament is administered once weekly.

The present invention provides methods for treating, preventing or delaying high blood pressure, comprising administering an effective amount of tirzepatide, or a pharmaceutically acceptable salt thereof to a patient in need of such treatment.

In an embodiment, the patient in need of treatment for high blood pressure has type 2 diabetes and is non-obese.

In an embodiment, the patient in need of treatment for high blood pressure has type 2 diabetes and obesity.

In an embodiment, the patient in need of treatment for high blood pressure has refractory type 2 diabetes.

In an embodiment, the patient in need of treatment for high blood pressure has type 2 diabetes for at least 8 years.

Accordingly, an embodiment provides a method of treating, preventing or delaying development of hypertensive crisis in a patient with refractory type 2 diabetes, comprising administering an effective amount of tirzepatide, or a pharmaceutically acceptable salt thereof, to the patient once weekly.

In an embodiment, the patient in need of treatment for hypertensive crisis has type 2 diabetes and is non-obese.

In an embodiment, the patient in need of treatment for hypertensive crisis has type 2 diabetes and obesity.

In an embodiment, the patient in need of treatment for hypertensive crisis has refractory type 2 diabetes.

In an embodiment, the patient in need of treatment for hypertensive crisis has type 2 diabetes for at least 8 years.

Accordingly, an embodiment provides a method of treating, preventing or delaying development of low HDL-C in a patient, comprising administering an effective amount of tirzepatide, or a pharmaceutically acceptable salt thereof, to the patient once weekly.

In another aspect, the present invention provides a method of preventing or delaying hypertensive crisis in a patient, comprising administering an effective amount of tirzepatide, or a pharmaceutically acceptable salt thereof, to the patient once weekly.

In another aspect, the present invention provides a method of preventing or delaying low HDL-C in a patient, comprising administering an effective amount of tirzepatide, or a pharmaceutically acceptable salt thereof, to the patient once weekly.

In another aspect, the present invention provides a method of treating high blood pressure in a patient receiving clinical treatment for type 2 diabetes using oral antidiabetic agents for at least 1 year, 2 years, 3, year, 4 year or 5 years wherein the patient's HbA1c is >7%, comprising administering an effective amount of tirzepatide, or a pharmaceutically acceptable salt thereof, to the patient once weekly.

In another aspect, the present invention provides a method of improving glycemic control in a patient with type 2 diabetes mellitus and at risk for high blood pressure, comprising administering an effective amount of tirzepatide, or a pharmaceutically acceptable salt thereof, to the patient once weekly, wherein the method provides a reduction in the risk of the patient experiencing hypertensive crisis.

In another aspect, the present invention provides a method of improving glycemic control in a patient with type 2 diabetes mellitus and at risk for high blood pressure, comprising administering an effective amount of tirzepatide, or a pharmaceutically acceptable salt thereof, to the patient once weekly for at least 30 weeks, wherein the method provides a reduction in the risk of the patient experiencing hypertensive crisis.

In another aspect, the present invention provides a method of improving weight management in a patient with obesity and at risk for high blood pressure, comprising administering an effective amount of tirzepatide, or a pharmaceutically acceptable salt thereof, to the patient once weekly, wherein the method provides a reduction in the risk of the patient experiencing hypertensive crisis.

In another aspect, the present invention provides a method for treating high blood pressure in patient, comprising administering an effective amount of tirzepatide, or a pharmaceutically acceptable salt thereof, to the patient once weekly, wherein the patient's weight is within a normal weight range for the patient.

In another aspect, the present invention provides a method of improving weight management in a patient with obesity and at risk for high blood pressure, comprising administering an effective amount of tirzepatide, or a pharmaceutically acceptable salt thereof, to the patient once weekly, wherein the method provides a reduction in the risk of the patient experiencing high blood pressure.

In another aspect, the present invention provides tirzepatide, or a pharmaceutically acceptable salt thereof, for use in treating, preventing or delaying development of hypertensive crisis, wherein the tirzepatide, or pharmaceutically acceptable salt thereof, is administered once weekly.

In another aspect, there is provided tirzepatide, or a pharmaceutically acceptable salt thereof, for use in treating, preventing or delaying development of high blood pressure, wherein the tirzepatide, or pharmaceutically acceptable salt thereof, is administered once weekly.

In another aspect, an embodiment is the use of tirzepatide, or a pharmaceutically acceptable salt thereof, for the preparation of a medicament for treating, preventing or delaying development of high blood pressure, wherein the medicament is administered once weekly.

In another aspect, provides the use of tirzepatide for the preparation of a medicament for treating, preventing or delaying development of high blood pressure, wherein the medicament is administered once weekly.

In another aspect, an embodiment is the use of tirzepatide, or a pharmaceutically acceptable salt thereof, for the preparation of a medicament for treating, preventing or delaying development of hypertensive crisis, wherein the medicament is administered once weekly.

In another aspect, provides the use of tirzepatide for the preparation of a medicament for treating, preventing or delaying development of hypertensive crisis, wherein the medicament is administered once weekly.

U.S. Pat. No. 9,474,780 describes and claims tirzepatide. When used herein, the term “tirzepatide” refers to any GIP/GLP-1 receptor agonist having the amino acid sequence of SEQ ID NO:1, including any protein that is the subject of a regulatory submission seeking approval of a GIP/GLP-1 receptor agonist product which relies in whole or part upon data submitted to a regulatory agency by Eli Lilly and Company relating to tirzepatide, regardless of whether the party seeking approval of said protein actually identifies the protein as tirzepatide or uses some other term. Tirzepatide agonizes the GIP/GLP-1 receptors resulting in stimulation of insulin synthesis and secretion, and has been shown to provide improved glycemic control in T2DM patients.

As used herein, “hypertensive crisis” means blood pressure is dangerously high and may threaten patient organs or life. Hypertensive crisis is typically blood pressure that is at least 180/120. High blood pressure is generally 130/80 systolic/diastolic pressure.

As used herein, “refractory type 2 diabetes” refers to a patient unable to achieve their HbA_{1c} goal using oral standard of care medications, such as metformin.

As used herein, “HbA_{1c} goal” means the desired average HbA_{1c} level to be achieved by the patient, as determined by the patient's clinical treatment plan and measured using clinically accepted methods. Current ADA guidelines suggest a reasonable HbA_{1c} treatment goal of less than or equal to 7% following current treatment options of diet, exercise, metformin, oral diabetes treatments, followed by basal insulin. However, many patients fail to reach their HbA_{1c} goals despite clinical treatment and are considered to have refractory type 2 diabetes. In an embodiment, the HbA_{1c} goal is 7% or less. In an embodiment, the HbA_{1c} goal is 5.7% or less.

Among men and women aged 49 to 82 yr, who were free of CHD at baseline in the Framingham study (during a follow up period of 12 yr), the participants with high HDL-C levels (in the 80^{th} percentile) were at 50 percent lower risk of cardiovascular events compared with participants with low HDL-C levels (in the 20^{th} percentile)². In the Prospective Cardiovascular Münster (PROCAM) study (˜4,500 volunteers, aged 16-65 yr, followed up for 6 yr), individuals with HDL-C<35 mg/dl were found to have four times higher coronary risk than those with HDL-C≥35 mg/dl. See, Assmann G et al., High-density lipoprotein cholesterol as a predictor of coronary heart disease risk. The PROCAM experience and pathophysiological implications for reverse cholesterol transport. Atherosclerosis. 1996; 124(Suppl):S11-S20. Gordon et al., suggested that every 1 mg/dl increase in HDL-C results in a decrease of 2 to 3 percent of composite cardiovascular risk to an individual. Gordon D J et al. High-density lipoprotein cholesterol and cardiovascular disease: four prospective American studies. Circulation. 1989; 79:8-15. Results from Veterans Administration HDL Intervention Trial (VA-HIT) showed that in patients with initially low HDL-C, a modest increase of only 6 percent of HDL-C significantly reduced both coronary morbidity and mortality up to 24 percent. Rubins H B, et al., Gemfibrozilfor the secondary prevention of coronary heart disease in men with low levels of high-density lipoprotein cholesterol. Veterans Affairs High-Density Lipoprotein Cholesterol Intervention Trial Study Group. N Engl J Med. 1999; 341:410-8.

Current treatments to increase HDL-C may include lifestyle modification such as increased exercise and decrease dietary fat. Often lifestyle modification is insufficient to attain desired increase HDL-C. There is a need for treatment options for a patient in need of HDL-C elevation. There is a need for therapies to treat, prevent, or delay low HDL-C.

When used herein, the terms “treatment,” “treat,” “treating,” and the like, are meant to include slowing or attenuating the progression of a disease, condition or disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

When used herein, the terms “prevent,” “preventing,” “prevention,” and the like, are meant to include avoidance of the onset of a disease, condition, disorder or symptom.

When used herein, the terms “delay,” “delaying,” and the like, are meant to include increasing the duration of time that occurs until onset of a disease, condition, disorder or symptom.

When used herein in connection with multiple outcomes, the term “composite” refers to the first to occur of any of the outcomes.

The term “increase HDL-C” means the measured HDL-C level increases from baseline. In an embodiment, increase HDL-C change is statistically significant increase. In an embodiment, increase HDL-C is greater than 2% increase from baseline. In an embodiment, increase HDL-C is greater than 5% increase from baseline. In an embodiment, increase HDL-C is greater than 7% increase from baseline. In an embodiment, increase HDL-C is greater than 10% increase from baseline.

“Therapeutically effective amount” means the amount of tirzepatide, or a pharmaceutically acceptable salt thereof, for the methods and uses of the present invention or pharmaceutical composition comprising tirzepatide, or a pharmaceutically acceptable salt thereof, for the methods and uses of the present invention that will elicit the biological or medical response of or desired therapeutic effect on the patient that is being sought by the researcher, medical doctor, or other clinician. An effective amount of tirzepatide, or a pharmaceutically acceptable salt thereof, may vary according to factors such as the disease state, age, sex, and weight of the individual, and the ability of tirzepatide to elicit a desired response in the individual. An effective amount is also one in which any toxic or detrimental effect is outweighed by the therapeutically beneficial effects. In certain embodiments, the therapeutically effective amount of tirzepatide, or a pharmaceutically acceptable salt thereof, for use in the methods described herein is selected from the group consisting of 5, 10 and 15 mg. In certain embodiments, the therapeutically effective amount of tirzepatide, or a pharmaceutically acceptable salt thereof, is 5.0 mg. In certain embodiments, the therapeutically effective amount of tirzepatide, or a pharmaceutically acceptable salt thereof, is 10.0 mg. In certain embodiments, the therapeutically effective amount of tirzepatide, or a pharmaceutically acceptable salt thereof, is 15.0 mg.

Additional embodiments are described below:

In an embodiment, a method of improving glycemic control and increasing HDL-C, in a patient with type 2 diabetes mellitus, comprising administering tirzepatide, or a pharmaceutically acceptable salt thereof, in a therapeutically effective amount to the patient once weekly for at least 30 weeks.

In an embodiment, tirzepatide, or a pharmaceutically acceptable salt thereof, is administered for at least 40 weeks. In an embodiment, tirzepatide, or a pharmaceutically acceptable salt thereof, is administered for at least 52 weeks.

### High Blood Pressure and Normal HbA_{1c} Glycemia

A method of treating, preventing or delaying development of high blood pressure in a patient, comprising administering tirzepatide, or a pharmaceutically acceptable salt thereof, in a therapeutically effective amount to the patient once weekly. In an embodiment, the high blood pressure is selected from the group consisting of high blood pressure and hypertensive crisis.

A method of preventing or delaying high blood pressure in a patient, comprising administering tirzepatide, or a pharmaceutically acceptable salt thereof, in a therapeutically effective amount to the patient once weekly.

A method of improving glycemic control and treating, preventing or delaying high blood pressure in a patient in a patient diagnosed with type 2 diabetes mellitus, comprising administering tirzepatide, or a pharmaceutically acceptable salt thereof, in a therapeutically effective amount to the patient once weekly.

In an embodiment, the method results in a reduction in the risk of the patient experiencing high blood pressure. In an embodiment, the method results in a reduction in the risk of the patient experiencing hypertensive crisis. In an embodiment, the method results in a reduction in the risk of the patient experiencing clinically low HDL-C.

A method of improving glycemic control in a patient with type 2 diabetes mellitus, comprising administering tirzepatide, or a pharmaceutically acceptable salt thereof, in a therapeutically effective amount to the patient once weekly, wherein the method results in a reduction in the risk of the patient experiencing high blood pressure.

The method of any of the above embodiments wherein the patient has type 2 diabetes mellitus. A method of any of the above embodiments wherein the patient has refractory type 2 diabetes. A method of any of the above embodiments wherein the patient has type 2 diabetes for at least 8 years. A method of any of the above embodiments wherein the patient has type 2 diabetes for at least 10 years. A method of any of the above embodiments wherein the once weekly administration of tirzepatide continues for at least 40 weeks. A method of any of the above embodiments wherein the patient is non-obese.

The method of any of the above embodiments wherein the patient has one or more of: T2DM; high blood pressure; reduced HDL-C; and obesity.

In an embodiment, the patient has either: multiple cardiovascular risk factors without high blood pressure or clinically significant high blood pressure.

In an embodiment, the patient has either: multiple cardiovascular risk factors or HbA1c level above 11%.

As used herein, “cardiovascular risk factors” means risk for cardiovascular disease selected from the group consisting of: current tobacco use (any form of tobacco); use of at least 1 approved lipid modifying therapy to treat hypercholesterolemia or a documented untreated low-density lipoprotein cholesterol (LDL-C)≥23.4 mmol/L (100 mg/dL) within the past 6 months; documented treated or untreated high-density lipoprotein cholesterol (HDL-C)<1.0 mmol/L (40 mg/dL) for men and <1.3 mmol/L (50 mg/dL) for women or triglycerides≥2.3 mmol/L (150 mg/dL) within the past 6 months; use of at least 1 blood pressure medication to treat high blood pressure or untreated systolic blood pressure (SBP)≥130 mm Hg or diastolic blood pressure (DBP)≥80 mmHg; measured waist circumference for a male 102 cm; for a female 88 cm.

As used herein, “non-obese” means a patient who is not obese by applicable standards. In an embodiment, the non-obese patient has body mass index is less than 30 BMI.

As used herein “comorbid” means that a patient is diagnosed with having 2 or more medical conditions.

In an embodiment, the patient's risk of hypertensive crisis is reduced by at least about 14%.

In an embodiment, the patient's risk of hypertensive crisis is reduced by at least about 10%.

In an embodiment, the HDL-C levels are increased. In an embodiment, HDL-C levels are increased to a clinically desired level. In an embodiment, is a method of improving glycemic control and increasing HDL-C, in a patient with type 2 diabetes mellitus, comprising administering tirzepatide, or a pharmaceutically acceptable salt thereof, in a therapeutically effective amount to the patient once weekly for at least 30 weeks.

In an embodiment, the risk of the occurrence of a composite of the following outcomes is reduced: hospitalization for high blood pressure or death.

In an embodiment, the risk of death or hospitalization for high blood pressure is reduced in a patient treated with an effective amount of tirzepatide, or a pharmaceutically acceptable salt thereof.

In an embodiment, the risk of the occurrence of a composite of the following outcomes is reduced: high blood pressure and HbA1c above 5.7%. In an embodiment, the risk of the occurrence of a composite of the following outcomes is reduced: low HDL-C, high blood pressure, and HbA1c above 7%.

In an embodiment, the risk of the occurrence of a composite of the following outcomes is reduced: low HDL-C, high blood pressure and HbA1c above 5.7%.

In an embodiment, the risk of the occurrence of a composite of the following outcomes is reduced: low HDL-C, high blood pressure, and HbA1c above 6%.

A method of reaching normal HbA1c glycemia in a patient with refractory type 2 diabetes comprising administering tirzepatide, or a pharmaceutically acceptable salt thereof, in a therapeutically effective amount to the patient once weekly.

In an embodiment, the amount of tirzepatide is selected from the group consisting of about 5.0 mg, about 10.0 mg and about 15.0 mg. In an embodiment, the amount of tirzepatide is selected from the group consisting of about 7.5 mg and about 12.5 mg.

In an embodiment, the amount of tirzepatide is about 5.0 mg.

In an embodiment, the amount of tirzepatide is about 10.0 mg.

In an embodiment, the amount of tirzepatide is about 15.0 mg.

In an embodiment, the dose of tirzepatide is about 5.0 mg.

In an embodiment, the dose of tirzepatide is about 10.0 mg.

In an embodiment, the dose of tirzepatide is about 15.0 mg.

In an embodiment, tirzepatide, or a pharmaceutically acceptable salt thereof, is administered using a dose escalation protocol.

In an embodiment, the patient fails to achieve HbA1C<7% using one or two oral diabetes agents for at least one year prior to treatment using tirzepatide, or pharmaceutically acceptable salt thereof.

In an embodiment, the patient fails to achieve HbA1C<8% using one or two oral agents for at least one year prior to treatment using tirzepatide, or pharmaceutically acceptable salt thereof.

In an embodiment, the patient fails to achieve HbA1C<10% using one or two oral agents for at least one year prior to treatment using tirzepatide, or pharmaceutically acceptable salt thereof.

In an embodiment, once weekly tirzepatide, or pharmaceutically acceptable salt thereof, administration continues for at least 30 weeks. In an embodiment, once weekly tirzepatide, or pharmaceutically acceptable salt thereof, administration continues for at least 40 weeks. In an embodiment, once weekly tirzepatide, or pharmaceutically acceptable salt thereof, administration continues for at least 50 weeks. In an embodiment, once weekly tirzepatide, or pharmaceutically acceptable salt thereof, administration continues for at least 2 years. In an embodiment, once weekly tirzepatide, or pharmaceutically acceptable salt thereof, administration continues for at least 3 years. In an embodiment, once weekly tirzepatide, or pharmaceutically acceptable salt thereof, administration continues for at least 5 years.

In an embodiment, the tirzepatide dose is 15 mg per week. In an embodiment, the tirzepatide dose is 10 mg per week. In an embodiment, the tirzepatide dose is 5 mg per week.

In an embodiment, the patient was diagnosed with type 2 diabetes is at least 8 years prior to tirzepatide, or pharmaceutically acceptable salt thereof, administration.

In an embodiment, the patient administered tirzepatide, or a pharmaceutically acceptable salt thereof, was diagnosed with type 2 diabetes is at least 10 years prior to tirzepatide administration.

In an embodiment, the patient administered tirzepatide, or a pharmaceutically acceptable salt thereof, was diagnosed with type 2 diabetes is at least 13 years prior to tirzepatide administration.

In an embodiment, the patient administered tirzepatide, or a pharmaceutically acceptable salt thereof, is at least 46 years old.

In an embodiment, the patient administered tirzepatide, or a pharmaceutically acceptable salt thereof, is at least 55 years old.

In an embodiment, the patient administered tirzepatide, or a pharmaceutically acceptable salt thereof, is at least 60 years old.

In an embodiment, the patient administered tirzepatide, or a pharmaceutically acceptable salt thereof, is also administered metformin and an SGLT2 oral agent.

In an embodiment, the patient administered tirzepatide, or a pharmaceutically acceptable salt thereof, is also administered an SGLT2 oral agent.

In an embodiment, the patient administered tirzepatide, or a pharmaceutically acceptable salt thereof, is also administered metformin.

In an embodiment, the patient administered tirzepatide, or a pharmaceutically acceptable salt thereof, is also administered a basal insulin.

In an embodiment, the patient administered tirzepatide, or a pharmaceutically acceptable salt thereof, is also administered metformin and a basal insulin.

In an embodiment the patient administered tirzepatide, or a pharmaceutically acceptable salt thereof, is also administered an SGLT2 and a basal insulin.

In an embodiment the patient administered tirzepatide, or a pharmaceutically acceptable salt thereof, is also administered metformin, an SGLT2, and a basal insulin.

In an embodiment, the basal insulin is insulin glargine.

In an embodiment, the basal insulin is insulin Degludec.

In an embodiment, the patient administered tirzepatide, or a pharmaceutically acceptable salt thereof, is also administered an SGLT2 oral pharmaceutical.

In an embodiment, the patient administered tirzepatide, or a pharmaceutically acceptable salt thereof, is also administered metformin, an SGLT2 oral, and insulin Degludec.

In an embodiment, the patient administered tirzepatide, or a pharmaceutically acceptable salt thereof, is also administered metformin, an SGLT2 oral, and insulin Degludec.

In an embodiment, the patient administered tirzepatide, or a pharmaceutically acceptable salt thereof, is also administered metformin, an SGLT2 oral, and inulin glargine.

In an embodiment, the patient administered tirzepatide, or a pharmaceutically acceptable salt thereof, is also administered metformin and insulin glargine.

Tirzepatide, or a pharmaceutically acceptable salt thereof, for use in any of the above embodiments.

Use of tirzepatide, or a pharmaceutically acceptable salt thereof, in the preparation of a medicament for any of the above embodiments.

Further embodiments are described in the examples below, which are not to be construed as limiting.

An embodiment provides tirzepatide, or a pharmaceutically acceptable salt thereof, for use in treating, preventing, or delaying high blood pressure in a patient, wherein the tirzepatide, or a pharmaceutically acceptable salt thereof, is administered once weekly. A further embodiment, provides tirzepatide, or a pharmaceutically acceptable salt thereof, for treating high blood pressure in a patient diagnosed with type 2 diabetes, wherein the tirzepatide, or a pharmaceutically acceptable salt thereof, is administered once weekly.

In another embodiment, the present invention provides tirzepatide, or a pharmaceutically acceptable salt thereof, for use in preventing, or delaying high blood pressure in a patient, wherein the tirzepatide, or a pharmaceutically acceptable salt thereof, is administered once weekly. In an embodiment, the patient is diagnosed with type 2 diabetes. In a further embodiment, the patient has type 2 diabetes and is non-obese. In an alternate embodiment, the patient has type 2 diabetes and obesity. In a further embodiment, the patient has refractory type 2 diabetes. In yet a further embodiment, the patient has type 2 diabetes for at least 8 years.

In another embodiment, the present invention provides tirzepatide, or a pharmaceutically acceptable salt thereof, for use in treating, preventing or delaying development of hypertensive crisis in a patient, wherein the tirzepatide, or a pharmaceutically acceptable salt thereof, is administered once weekly. In an embodiment, the patient is diagnosed with type 2 diabetes. In a further embodiment, the patient has type 2 diabetes and is non-obese. In an alternate embodiment, the patient has type 2 diabetes and obesity. In a further embodiment, the patient has refractory type 2 diabetes. In yet a further embodiment, the patient has type 2 diabetes for at least 8 years.

In another embodiment, the present invention provides tirzepatide, or a pharmaceutically acceptable salt thereof, for use in treating, preventing or delaying development of low HDL-C in a patient, wherein the tirzepatide, or a pharmaceutically acceptable salt thereof, is administered once weekly.

In another embodiment, the present invention provides tirzepatide, or a pharmaceutically acceptable salt thereof, for use in preventing or delaying development of low HDL-C in a patient, wherein the tirzepatide, or a pharmaceutically acceptable salt thereof, is administered once weekly.

In another embodiment, the present invention provides tirzepatide, or a pharmaceutically acceptable salt thereof, for use in treating high blood pressure in a patient receiving clinical treatment for type 2 diabetes using oral antidiabetic agents for at least 1 year, 2 years, 3, year, 4 year or 5 years, wherein the patient's HbA1c is >7% and wherein the tirzepatide, or a pharmaceutically acceptable salt thereof, is administered once weekly.

In another embodiment, the present invention provides tirzepatide, or a pharmaceutically acceptable salt thereof, for use in improving glycemic control in a patient with type 2 diabetes mellitus and at risk for high blood pressure, wherein the tirzepatide, or a pharmaceutically acceptable salt thereof, is administered once weekly. In an embodiment, the tirzepatide, or a pharmaceutically acceptable salt thereof, is administered once weekly for at least 30 weeks.

In another embodiment, the present invention provides tirzepatide, or a pharmaceutically acceptable salt thereof, for use in improving weight management in a patient with obesity and at risk for high blood pressure, wherein the tirzepatide, or a pharmaceutically acceptable salt thereof, is administered once weekly.

In another embodiment, the present invention provides tirzepatide, or a pharmaceutically acceptable salt thereof, for use in treating high blood pressure in patient, wherein the tirzepatide, or a pharmaceutically acceptable salt thereof, is administered once weekly and wherein the patient's weight is within a normal weight range for the patientIn other embodiments, present invention provides the following:
Embodiment 1. Tirzepatide, or a pharmaceutically acceptable salt thereof, for use in the treatment of refractory type 2 diabetes in a patient, wherein the tirzepatide, or a pharmaceutically acceptable salt thereof, is administered once weekly.
Embodiment 2. Tirzepatide, or a pharmaceutically acceptable salt thereof, for use in the treatment of high blood pressure in a patient, wherein the tirzepatide, or a pharmaceutically acceptable salt thereof, is administered once weekly.
Embodiment 3. Tirzepatide, or a pharmaceutically acceptable salt thereof, for use in raising HDL-C in a patient, wherein the tirzepatide, or a pharmaceutically acceptable salt thereof, is administered once weekly.
Embodiment 4. Tirzepatide, or a pharmaceutically acceptable salt thereof, for use according to any one of embodiments 1 to 3, wherein the patient has type 2 diabetes for at least 8 years.
Embodiment 5. Tirzepatide, or a pharmaceutically acceptable salt thereof, for use according to any one of embodiments 1 to 4, wherein the patient HbA_{1c} goal is less than 7%.
Embodiment 6. Tirzepatide, or a pharmaceutically acceptable salt thereof, for use according to any one of embodiments 1 to 5, wherein the patient HbA_{1c} goal is equal to or less than 5.7%.
Embodiment 7. Tirzepatide, or a pharmaceutically acceptable salt thereof, for use according to any one of embodiments 1 to 6, wherein the patient HbA_{1c} is greater than 10%.
Embodiment 8. Tirzepatide, or a pharmaceutically acceptable salt thereof, for use according to any one of embodiments 1 to 7, wherein the patient HbA_{1c} is greater than 11%.
Embodiment 9. Tirzepatide, or a pharmaceutically acceptable salt thereof, for use according to any one of embodiments 1 to 8, wherein the patient age is at least 46 years.
Embodiment 10. Tirzepatide, or a pharmaceutically acceptable salt thereof, for use according to any one of embodiments 1 to 9, wherein the patient age is at least 60 years old.
Embodiment 11. Tirzepatide, or a pharmaceutically acceptable salt thereof, for use according to any one of embodiments 1 to 10, wherein the patient is taking an SGLT2 inhibitor.
Embodiment 12. Tirzepatide, or a pharmaceutically acceptable salt thereof, for use according to any one of embodiments 1 to 11, wherein the patient is taking metformin.
Embodiment 13. Tirzepatide, or a pharmaceutically acceptable salt thereof, for use according to any one of embodiments 1 to 12, wherein the patient is not administered a basal insulin.
Embodiment 14. Tirzepatide, or a pharmaceutically acceptable salt thereof, for use according to any one of embodiments 1 to 13, wherein the patient fails to reach their HbA_{1c} goal while taking metformin and an SGLT2 inhibitor.
Embodiment 15. Tirzepatide, or a pharmaceutically acceptable salt thereof, for use according to any one of embodiments 1 to 14, wherein the tirzepatide, or a pharmaceutically acceptable salt thereof, is administered for at least 40 weeks.
Embodiment 16. Tirzepatide, or a pharmaceutically acceptable salt thereof, for use according to any one of embodiments 1 to 15, wherein the tirzepatide, or a pharmaceutically acceptable salt thereof, is administered for at least 50 weeks.
Embodiment 17. Tirzepatide, or a pharmaceutically acceptable salt thereof, for use according to any one of embodiments 1 to 16, wherein the tirzepatide, or a pharmaceutically acceptable salt thereof, is administered for at least 2 years.
Embodiment 18. Tirzepatide, or a pharmaceutically acceptable salt thereof, for use according to any one of embodiments 1 to 17, wherein the patient is non-obese.
Embodiment 19. Tirzepatide, or a pharmaceutically acceptable salt thereof, for use according to any one of embodiments 1 to 18, wherein the once weekly dose of tirzepatide, or a pharmaceutically acceptable salt thereof, is 5 mg.
Embodiment 20. Tirzepatide, or a pharmaceutically acceptable salt thereof, for use according to any one of embodiments 1 to 18, wherein the once weekly dose of tirzepatide, or a pharmaceutically acceptable salt thereof, is 10 mg.
Embodiment 21. Tirzepatide, or a pharmaceutically acceptable salt thereof, for use according to any one of embodiments 1 to 18, wherein the once weekly dose of tirzepatide, or a pharmaceutically acceptable salt thereof, is 15 mg.
Embodiment 22. Tirzepatide, or a pharmaceutically acceptable salt thereof, for use according to any one of embodiments 1 to 21, wherein the patient has comorbid high blood pressure.
Embodiment 23. Tirzepatide, or a pharmaceutically acceptable salt thereof, for use according to any one of embodiments 1 to 22, wherein the patient has comorbid low HDL-C.
Embodiment 24. Tirzepatide, or a pharmaceutically acceptable salt thereof, for use according to any one of embodiments 1 to 17 or 19 to 22, wherein the patient has comorbid obesity.
Embodiment 25. Tirzepatide, or a pharmaceutically acceptable salt thereof, for use according to any one of embodiments 1 to 21, wherein the patient has at least two cardiovascular risk factors.
Embodiment 26. Tirzepatide, or a pharmaceutically acceptable salt thereof, for use according to any one of embodiments 1 to 21, wherein the patient has no cardiovascular risk factors.
Embodiment 27. Tirzepatide, or a pharmaceutically acceptable salt thereof, for use according to any one of embodiments 1 to 26, wherein the patient has type 2 diabetes for at least 10 years.

As used herein “estimands” refers to—efficacy and treatment-regimen—evaluated to determine the efficacy of tirzepatide due to requirements by certain regulatory agencies. The efficacy estimand is used to evaluate results in people prior to their discontinuation of study drug or initiating rescue therapy for persistent severe hyperglycemia. The treatment-regimen estimand—required by certain regulatory agencies including the U.S. Food and Drug Administration—evaluates the treatment effect in people in the study irrespective of adherence to tirzepatide or introduction of rescue therapy for persistent severe hyperglycemia.

### EXAMPLES

### Example 1. Clinical Trial Using Tirzepatide

The enrollment criteria, set forth in Table 1 below, are designed to include participants who are similar to patients seen within a typical diabetes practice, and include some patient with type 2 diabetes that is refractory to oral treatment. Geriatric patients are included. Mean duration with type 2 diabetes about 8 years. Study continues for 52 weeks.

TABLE 1
Enrollment Criteria Example 1.
Key inclusion criteria

T2DM with HbA1c between ≥7.0% and ≤10.5%
Be on stable treatment with unchanged dose of metformin or metformin plus an SGLT-
2 inhibitor for at least 3 months before screening
Be of stable weight (±5%) for at least 3 months before screening
Have a BMI ≥25 kilograms per meter squared (kg/m2) at screening
Run-in adherence to study drug = 100%
Signed informed consent

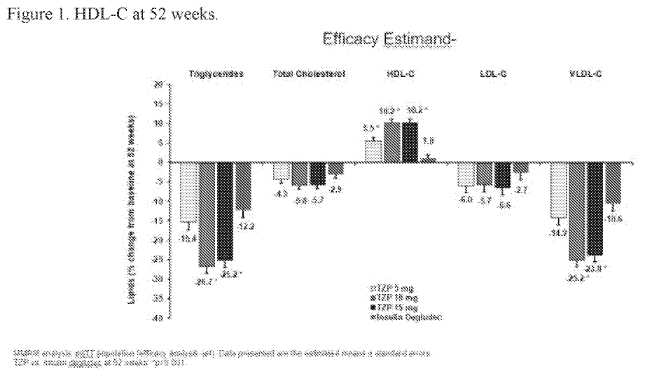

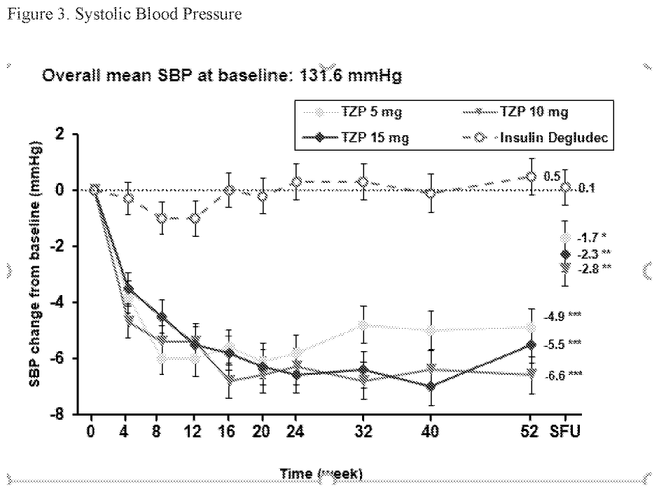

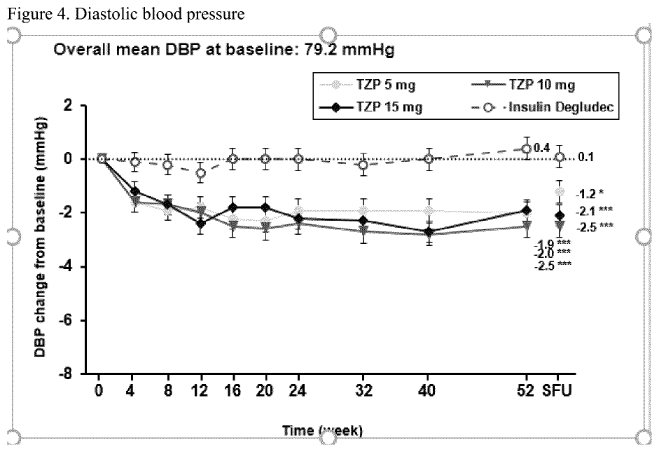

The study is designed to consist of a screening visit followed by a single-blind 3-week placebo run-in period. Afterwards, patients are randomized to tirzepatide 5, 10, or 15 mg (dosed using an escalation dose protocol) or insulin degludec (standard titration protocol) and followed at approximately monthly intervals. Patients receiving insulin degludec follow standard insulin degludec titration protocol during the study. Study protocol includes blood pressure measurement at each visit and serum lipid profile at study initiation and at 52 weeks using standard clinical methods. Blood Pressure measures using the methods of Example 1 are represented in FIGS. 3 and 4. HDL-C levels using the methods of Example 1 are represented in FIG. 1.

Up to 92.6 percent of participants receiving tirzepatide treatment achieved an HbA_{1c} of less than 7 percent, the American Diabetes Association's recommended target for people with diabetes. Up to 48.5 percent of participants receiving tirzepatide in the study achieved an HbA_{1c} less than 5.7 percent—the level seen in people without diabetes.

TABLE 2
Percent patients achieving ≤5.7% HbA_{1c} by 52 weeks

TZP 5 mg: 25.8%
TZP 10 mg: 38.6%
TZP 15 mg: 48.4%
insulin degludec: 5.4%

The mean dose of insulin degludec at 52 weeks is 48.8 units per day.

### Example 2. Clinical Trial Using Tirzepatide

The enrollment criteria, set forth in Table 3 below, include patients considered refractory to type 2 diabetes oral treatment; however, oral diabetes treatment continues through the study. Mean duration with type 2 diabetes about 13 years. Study continues for 40 weeks.

Clinical Trial 2, Table 3

TABLE 3
Enrollment Criteria Example 2.
Key inclusion criteria

T2DM with HbA1c between ≥7.0% and ≤10.5%
Be treated with insulin glargine (U100), once daily, with or without metformin or
metformin for at least 3 months before screening
Be of stable weight (±5%) for at least 3 months before screening
Have a BMI ≥23 kilograms per meter squared (kg/m2) at screening
Run-in adherence to study drug = 100%
Signed informed consent

The study is designed to consist of a screening visit followed by a 3-week run-in period. Afterwards, patients begin a 40-week randomized, double-blind study with tirzepatide 5, 10, or 15 mg or placebo, as add-on to their previous treatment with insulin glargine with or without metformin. Insulin glargine dose was titrated throughout the study using a validated treat-to-target algorithm'. Patients are followed at approximately weekly intervals, then approximately monthly. Study protocol includes blood pressure measurement at each visit and serum lipid profile at study initiation and at 40 weeks using standard clinical methods.

Three doses of tirzepatide (5 mg, 10 mg and 15 mg) demonstrated superior HbA_{1c} reductions and weight reductions from baseline compared to placebo as an add-on to titrated insulin glargine with or without metformin in adults with type 2 diabetes using the clinical trial dosing. Across three tirzepatide doses, up to 97.4 percent of participants achieved an HbA_{1c} of less than 7 percent. Further, 62.4 percent of participants treated with the highest tirzepatide dose achieved an HbA_{1c} less than 5.7 percent.

TABLE 4
Percent patients achieving 7% or less
HbA1c by 40 weeks with add-on.

Using the efficacy estimand:
TZP 5 mg: 93%
TZP 10 mg: 97.4%
TZP 15 mg: 94%
Placebo: 33.9%

TABLE 5
percent of patients achieving HbA1c level
of 5.7 or less by 40 weeks with add on.

Using the efficacy estimand:
TZP 5 mg: 26.1%
TZP 10 mg: 47.8%
TZP 15 mg: 62.4%
Placebo: 2.5%

HDL-C levels using the methods of Example 1 are represented in FIG. 1.

### Sequences

### SEQ ID NO:1

### Tirzepatide

YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with (2-[2-(2-Amino-ethoxy)-ethoxy]-acetyl)₂-(γGlu)₁-CO—(CH₂)₁₈—CO₂H; and the C-terminal amino acid is amidated as a C-terminal primary amide.

1. A method of treating refractory type 2 diabetes in a patient in need thereof, comprising administering an effective amount of tirzepatide, or a pharmaceutically acceptable salt thereof, to the patient once weekly.

2. A method as claimed by claim 1, wherein the patient has type 2 diabetes for at least 8 years.

3. A method as claimed by claim 1, wherein the patient HbA_{1c} goal is less than 7%.

4. A method as claimed by claim 1, wherein the patient HbA_{1c} goal is equal to or less than 5.7%.

5. A method as claimed by claim 1, wherein the patient HbA_{1c} is greater than 10%.

6. A method as claimed by claim 1, wherein the patient HbA_{1c} is greater than 11%.

7. A method as claimed by claim 1, wherein the patient age is at least 46 years.

8. A method as claimed by claim 1, wherein the patient age is at least 60 years old.

9. A method as claimed by claim 1, wherein the patient is taking an SGLT2 inhibitor.

10. A method as claimed by claim 1, wherein the patient is taking metformin.

11. A method as claimed by claim 1, wherein the patient is not administered a basal insulin.

12. A method as claimed by claim 1, wherein the patient fails to reach their HbA_{1c} goal while taking metformin and an SGLT2 inhibitor.

13. A method as claimed by claim 1, wherein the tirzepatide, or pharmaceutically acceptable salt thereof, treatment continues for at least 40 weeks.

14. A method as claimed by claim 1, wherein the patient is administered tirzepatide for at least 50 weeks.

15. A method as claimed by claim 1, wherein the patient is administered tirzepatide for at least 2 years.

16. A method as claimed by claim 1, wherein the patient is non-obese.

17. A method as claimed by claim 1 wherein the effective amount is 5 mg.

18. A method as claimed by claim 1, wherein the effective amount is 10 mg.

19. A method as claimed by claim 1, wherein the effective amount is 15 mg.

20. A method as claimed by claim 1, wherein the patient has comorbid high blood pressure.

21. A method as claimed by claim 1, wherein the patient has comorbid low HDL-C.

22. A method as claimed by claim 1, wherein the patient has comorbid obesity.

23. A method as claimed by claim 1, wherein the patient has at least two cardiovascular risk factors.

24. A method as claimed by claim 1, wherein the patient has no cardiovascular risk factors.

25. A method as claimed by claim 1, wherein the patient has type 2 diabetes for at least 10 years.

26. A method of treating high blood pressure in a patient in need thereof, comprising administering an effective amount of tirzepatide, or a pharmaceutically acceptable salt thereof, to the patient once weekly.

27. A method as claimed by claim 26, wherein the patient has type 2 diabetes for at least 8 years.

28. A method as claimed by claim 26, wherein the patient has refractory type 2 diabetes.

29. A method as claimed by claim 27, wherein the patient HbA_{1c} goal is less than 7%.

30. A method as claimed by claim 26, wherein the patient HbA_{1c} goal is equal to or less than 5.7%.

31. A method as claimed by claim 26, wherein the patient HbA_{1c} is greater than 10%.

32. A method as claimed by claim 26, wherein the patient HbA_{1c} is greater than 11%.

33. A method as claimed by claim 26, wherein the patient age is at least 46 years.

34. A method as claimed by claim 26, wherein the patient age is at least 60 years old.

35. A method as claimed by claim 26, wherein the patient is taking an SGLT2 inhibitor.

36. A method as claimed by claim 26, wherein the patient is taking metformin.

37. A method as claimed by claim 26, wherein the patient is not administered a basal insulin.

38. A method as claimed by claim 26, wherein the patient fails to reach their HbA_{1c} goal while taking metformin and an SGLT2 inhibitor.

39. A method as claimed by claim 26, wherein the tirzepatide treatment continues for at least 40 weeks.

40. A method as claimed by claim 26, wherein the patient has comorbid obesity.

41. A method as claimed by claim 26, wherein the patient is non-obese.

42. A method as claimed by claim 26, wherein the effective amount is 5 mg.

43. A method as claimed by claim 26, wherein the effective amount is 10 mg.

44. A method as claimed by claim 26, wherein the effective amount is 15 mg.

45. A method as claimed by claim 26, wherein the patient has comorbid low HDL-C.

46. A method as claimed by claim 26, wherein the patient has at least two cardiovascular risk factors.

47. A method as claimed by claim 26, wherein the high blood pressure is hypertensive crisis.

48. A method as claimed by claim 26, wherein the method prevents hypertensive crisis.

49. A method as claimed by claim 26, wherein the method prevents stroke.

50. A method for raising HDL-C in a patient in need thereof, comprising administering an effective amount of tirzepatide, or a pharmaceutically acceptable salt thereof, to the patient once weekly.

51. A method as claimed by claim 50, wherein the patient has type 2 diabetes for at least 8 years.

52. A method as claimed by claim 50, wherein the patient has refractory type 2 diabetes.

53. A method as claimed by claim 50, wherein the patient age is at least 46 years.

54. A method as claimed by claim 50, wherein the patient age is at least 60 years old.

55. A method as claimed by claim 50, wherein the patient is taking an SGLT2 inhibitor.

56. A method as claimed by claim 50, wherein the patient is taking metformin.

57. A method as claimed by claim 50, wherein the patient is not administered a basal insulin.

58. A method as claimed by claim 50, wherein the tirzepatide treatment continues for at least 40 weeks.

59. A method as claimed by claim 50, wherein the patient is non-obese.

60. A method as claimed by claim 50, wherein the effective amount is 5 mg.

61. A method as claimed by claim 50, wherein the effective amount is 10 mg.

62. A method as claimed by claim 50, wherein the effective amount is 15 mg.

63. A method as claimed by claim 50, wherein the patient has at least two cardiovascular risk factors.

64. A method as claimed by claim 50, wherein the patient has comorbid high blood pressure.

65. A method as claimed by claim 50, wherein the tirzepatide administration continues at least 50 weeks.

66. A method as claimed by claim 50, wherein once weekly administration of tirzepatide is continued for at least 2 years.


SEQUENCE LISTING (1 sequence)

SEQ ID NO: 1 - protein, 39 aa, Artificial Sequence
  Synthetic Construct
  MISC_FEATURE (2)..(2): Xaa at position 2 is nonnaturally occurring amino acid 2-Aminoisobutyric Acid
  MISC_FEATURE (13)..(13): Xaa at position 13 is nonnaturally occurring amino acid 2-Aminoisobutyric Acid
  MOD_RES (20)..(20): Lys at position 20 is chemically modified through conjugation to the epsilon-amino group of the K side-chain with (2-[2-(2-Amino-ethoxy)-ethoxy]-acetyl)2-(gamma-Glu)1- CO-(CH2)18-CO2H
  MOD_RES (39)..(39): Ser at position 39 is amidated as a C-terminal primary amide
YXEGTFTSDYSIXLDKIAQKAFVQWLIAGGPSSGAPPPS